(19) United States
(12) Patent Application Publication
(10) Pub. No.: US 2014/0235921 A1
(43) Pub. Date: Aug. 21, 2014

(54) SYSTEM AND METHOD FOR POSITIONING WITH NUCLEAR IMAGING

(71) Applicants: SurgicEye GmbH, München (DE); humediQ GmbH, Gräfelfing (DE)

(72) Inventors: Thomas Wendler, Munchen (DE); Christian Hieronimi, Schwabmunchen (DE); Jörg Traub, Munchen (DE)

(73) Assignees: SurgicEye GmbH, München (DE); humediQ GmbH, Gräfelfing (DE)

(21) Appl. No.: 14/346,539

(22) PCT Filed: Sep. 24, 2012
(86) PCT No.: PCT/EP2012/068759

§ 371 Date: Mar. 21, 2014

(30) Foreign Application Priority Data
Sep. 22, 2011 (DE) 1020110538682

(51) Int. Cl. A61N 5/10

(52) U.S. Cl. CPC A61N 5/1049; A61N 5/1064; A61N 2005/1052; A61N 2005/1059
USPC 600/1

(57) ABSTRACT

The invention relates to a system for determining the position of radioactively marked target tissue of a patient in radiation therapy by means of a radiation therapy device. The system comprises at least one detector for the imaging detection of the radiation distribution of a radioactive radiation source that is located in target tissue region, a computing unit that is designed to calculate the pose of the target tissue region from measurement data of the detector and to calculate a correction variable as the difference between the calculated pose and comparison values, and an interface by means of which the data for the calculated pose of radioactively marked target tissue region and the correction variable can be transmitted by the computing unit to a radiation therapy device or a patient positioning device. The invention further relates to a corresponding method.

The present disclosure relates to the field of nuclear imaging, such as PET or SPECT, and aspects pertain to systems and methods for determining the position of radioactively marked target tissue of a patient during radiation therapy with a radiation therapy device, and to a patient positioning device and methods for positioning a patient relative to a radiation source for radiation therapy, in dependence of measurement data of at least one nuclear radiation detector.

The therapy of tumors using various radiation types is one of the standard method in oncology. Using these therapy forms, an important subject is the highly selective radiation of the target tissue, as it is an aim to damage as little surrounding healthy tissue as possible. Therefore, a precise acquisition/recognition of the target structure, and based thereon an alignment of the radiation therapy device is an aim in the development of respective systems.

For positioning patients in a radiation room under a linear accelerator, a proton therapy system, or other external radiation devices, typically positioning systems are used. These include, e.g., fixation systems, e.g. for fixating single limbs, thermoplastical fixation masks, laser assisted positioning systems, robot based positioning systems, ultrasound based positioning systems etc.; and X-ray images are used, such as in multiple 2D X-ray positioning systems, Gantry-mounted primary beam computer tomographs, or megavolt computer tomographs, further optical positioning systems based on preoperative CT or MR data and on registration.

Positioning is a demanding task, because, e.g., the anatomy may change between radiation fractions, e.g. caused by organic changes in the tissue, air, water, or food enclosures, shrinking of the tumor, etc., wherein these changes occur on a timescale of days or weeks between the radiation fractions.

Another problem lies in the fact that even during treatment, the pose of the target area can vary at least to a small amount, e.g., caused by patient movement, for example minimal changes in the posture due to breathing, heartbeat, or coughing.

Therefore, a plurality of factors interact, which complicates the correct and sustainable positioning of a patient.

Previous approaches include, for example, the already mentioned X-ray based positioning systems, which can also be used during radiation treatment. Such systems are often equipped with movement sensors, which are able to detect larger changes, and subsequently trigger the acquisition of further X-ray images. Using these updated X-ray images, the patient may then be newly positioned or repositioned. This approach bears the disadvantage that it implies an X-ray radiation dose with each image. This means, amongst others, that it is not possible to acquire new images in real time—which implies that not only the additional radiation dose has to be seen as a disadvantage, but also the slow rate of repositioning of the patient.

A further approach includes the use of information which is acquired from the surface of the patient, in order to gather internal deformations and movements. Possible variants use markers which are provided on the skin (e.g. optical markers) or the surface of the patient itself, e.g. when using surface acquisition systems such as time of flight cameras, a stereotactic camera system or laser scanning systems. Such systems bear the disadvantage that they are not suitable to precisely acquire deformations and movements of deep lying structures, because the movements of the latter naturally are not directly connected to movements of the surface.

Alternatively, there are approaches where sensors are implanted into the target tissue in order to be able to track internal movements. Examples are systems provided by the companies Calypso and Navitek. In the first one, electromagnetic sensors are implanted in a prostate and are tracked during radiation treatment, for actively adapting the radiation. In the case of Navitek, a radioactive marker is implanted in the prostate and is localized by a collimator system in 3D, in order to adapt the radiation using this position. These systems bear the problem that they can only track those distinct implanted sensors. Technically, only two or at best three of these sensors can be tracked with sufficient accuracy. Complex structures and the shape of the tumors cannot be gathered, and hence the radiation can only be adapted suboptimal. Tumors which can marked systemically cannot be tracked at all with such systems.

For movements of a periodic nature such as those caused by heart beat or breathing, methods such as gating are used. In these methods it is only radiated when the patient is in a certain phase of breathing, the heart beat or both, in which the position of the target tissue is known. For the detection of this phase, often breathing sensors and heart beat sensors are used. However, non-periodical movements cannot be tracked by these systems. Further, these systems rely on the fact that breathing or heart beat is in fact periodical, which is, however, not the case.

In view of the above, a system for the determination of a position of a target tissue of a patient during radiation therapy with a radiation therapy device according to claim 1, and a respective method according to claim 11 are proposed. Further preferred aspects of the invention derive from the dependent claims, the drawings and the description.

According to a first aspect, a system for the determination of a position of a target tissue of a patient during radiation therapy with a radiation therapy device is provided. It includes at least one detector for the imaging of a radiation distribution of a radioactive source marking a target tissue region, a processing unit, adapted for calculating a pose of the target tissue region from measurement data of the at least one detector, as well as for calculating a correction value as a difference between the calculated pose and comparative data, an interface for transmitting data on the calculated pose of the target tissue region and the correction value from the processing unit to a radiation therapy device or to a patient positioning device.

According to a second aspect, a method for the determination of a position of a target tissue of a patient during radiation therapy with a radiation therapy device is provided. It includes applying a radioactive radiation source into the target tissue to be therapeutically radiated for marking it, imaging an emitted radiation distribution of the radiation source with at least one detector, while the patient is in a treatment position, calculation of a pose of the target tissue region using the detected radiation, calculation of the correction value as a difference between the calculated pose of the target tissue region and a setpoint value of the pose.

The invention further relates to an apparatus for carrying out the disclosed methods and includes also apparatus parts for carrying out single method steps. These method steps may be carried out by hardware components, by a computer programmed by respective software, by a combination of the former, or in any other manner. The invention is further also directed to methods according to which the disclosed apparatuses work. It includes method steps for carrying out every function of the apparatuses.

In the following, the invention shall be explained using exemplary embodiments shown in drawings, from which further advantages and modifications may be derived.

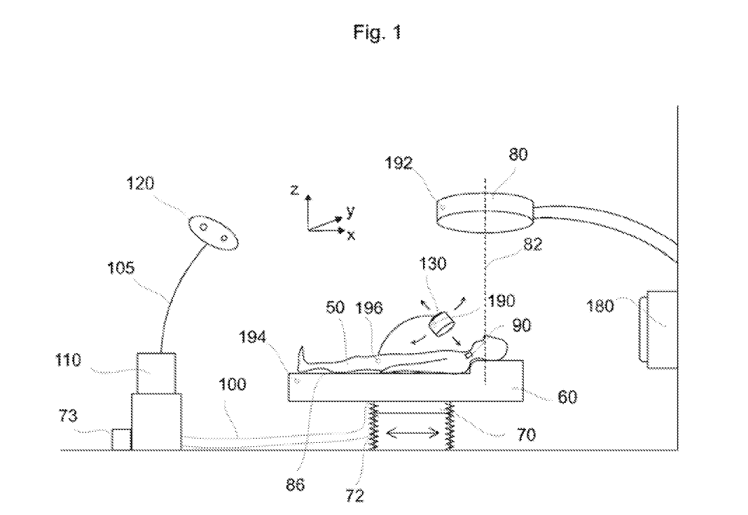

FIG. 1 shows a system for positioning according to embodiments of the invention;

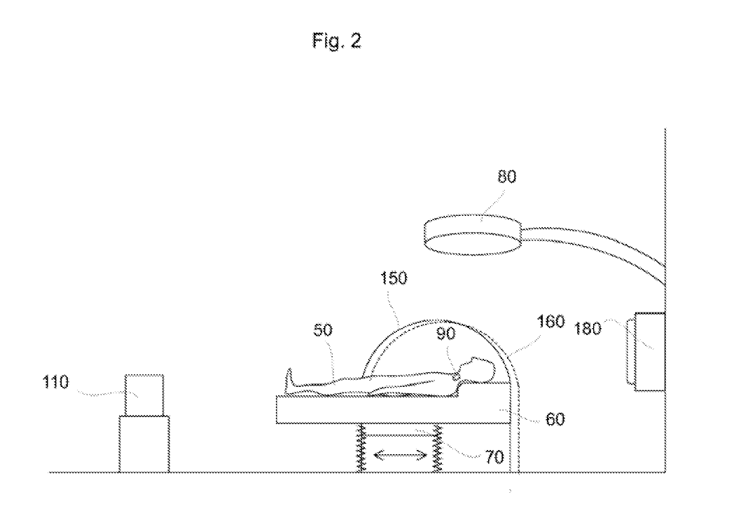

FIG. 2 shows a system for positioning according to further embodiments of the invention;

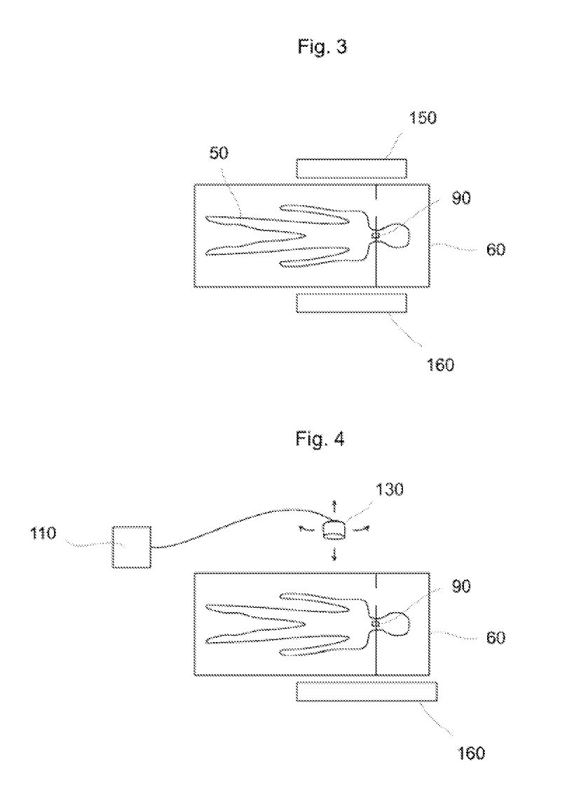

FIG. 3 shows a top view on a section of a system according to embodiments;

FIG. 4 shows a top view of a section of a system according to further embodiments;

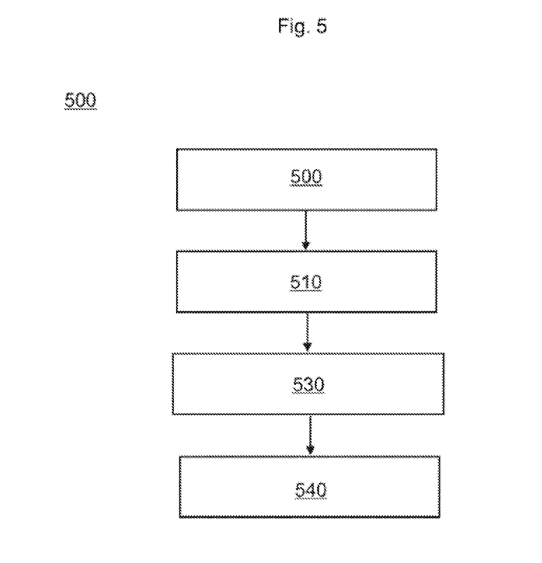

FIG. 5 shows a schematic view of a method according to embodiments.

In the following, various embodiments of the invention are described, of which some are also exemplarily depicted in the drawings. In the following description of the drawings, same reference numerals relates to identical or similar components. In general, only the differences between different embodiments are described. Thereby, features which are described as part of one embodiment may readily be combined with other embodiments in order to achieve further embodiments.

The structures to be radiated which are discussed herein (target structures, target tissue regions) are usually tumors or lymph nodes, which may be imaged with a radioactive tracer by nuclear medical imaging systems (PET, SPECT, gamma cameras, Compton cameras, freehand SPECT, etc.). Alternatively, structures which cannot be marked systemically or with a functional marker such as the sentinel marker, using nuclear medicine, may be marked directly by injection or implantation of radioactivity. Hence, these structures become radioactive and may be imaged employing nuclear medicine. A typical example is the implantation of an I-125 marker having a titanium cladding at the location of the tumor.

The term “PET detector” relates to any kind of coincidence camera system which includes at least two distinct detectors, which pertain to at least a part of the relevant anatomy on an imaginary line between the two detectors, and which are connected to a coincidence device for detecting coincidences (simultaneous detections in a single energy region) in both detectors.

The term “freehand SPECT detector” relates to any kind of freely movable, tracked detector (also non-imaging ones like gamma ray sondes), which are suitable to reconstruct a 3D image from measurements in different directions of a single photon radiation.

In embodiments, a nuclear medical picture of the target structure (e.g. the tumor) in the patient is produced prior to the radiation treatment in the radiation treatment room and is used for the initial positioning of the patient. Therefore, the patient is lying on the gantry of the radiation therapy device, or sits, if this is for example required by the design. The radiation coming from the marked tissue is acquired for a defined time span by a detector system having at least one detector, wherein the time span is typically in the range between 0.5 seconds and a few minutes, e.g. 3, 10, or 20 seconds. The time actually required for the measurements is, amongst other factors, dependent on the nature of the applied and detected radiation, the strength of the radiation source, how deep it is located in the tissue and is thus shielded, the sensitivity of the detector and the desired spatial resolution.

The detection system is so adapted, that information about the size, the position, and the shape of the target issue in space may be achieved by postprocessing of the acquired detector signals. Such a configuration may be achieved with a plurality of possible detector variants and combinations, wherein the required location in formation is, in some embodiments, achieved by a variation of the detector position during measurement, in the embodiments also by using collimators in front of the detector(s). The term “pose” as used herein, which is known to the skilled person, implicates that three space coordinates and two or three angle coordinates are determined (which define the orientation).

For calculating the position of the target structure from the detector data, basically known methods of image processing are applied. Amongst others, this includes segmentation, classification, and atlas, in order to determine a 3D position, shape and orientation of the target structure in space. Suitable methods for achieving location and shape information from detector data are known to the skilled person and shall not be discussed in detail.

By comparing this data with the geometric data of the radiation path of the radiation therapy device, it may be determined how the position of the patient has to be altered in order to bring the target tissue, e.g. the tumor, into the correct position. The determined correction or correction value is in the most simple case a three dimensional vector, the length and direction of which indicate the required change needed to begin with the radiation treatment. Using this correction vector, the patient may be positioned at the correct position for the radiation treatment. The correction value may also be a matrix (e.g. a rigid 4×4 transformation matrix), or particularly a deformation field, which is further described below.

The correction vector, respectively the correction value is displayed to an operator of the radiation therapy device for the purpose of re-positioning of the patient. This can have the form of a graphical representation on a display, e.g. a LCD monitor, or via an audio signal. Also possible is an overlay of the correction information with a video picture acquired by a camera.

Alternatively or additionally this correction may be fed into a positioning system, which automatically positions the patient into a correct position with respect to the radiation therapy device. This correction value may also be stored and may be used as an initial value for subsequent radiation fractions.

Further, the described imaging procedure may continue to generate images during a radiation session and may thus update the described correction vector in real time. This real-time update may be displayed to a user or may trigger an optical and or acoustical warning. Should the required, calculated correction be too large (that is should the position of the tumor deviate too much from the setpoint value), the radiation may be automatically stopped, for example by switching on or of an interlock. An interlock is a device which stops or interrupts the movements or the radiation of a radiation therapy device, if a patient, an operator, or the radiation therapy device itself might suffer a damage.

Further, the correction value may be used to start the radiation when it falls below a certain value. Further, the correction value may be fed into a positioning system for automatically carrying out a correction during radiation therapy.

In embodiments, the method described above includes at least one detector, a processing unit for data processing, and an interface for transmitting data between the processing unit and the radiation therapy device. In embodiments, the processing unit may also be provided as a part of the radiation therapy device, so that the only unit required additional to the radiation therapy device is the one or or more detector units.

FIG. 1 shows the system according to an embodiment. The patient 50 lies on a treatment couch 60. It is connected with a patient positioning device 70, which may adjust the treatment couch vertically and horizontally in several directions, for changing the position of the patient with respect to the beam of radiation therapy device 80. Previous to the treatment, the radiation source 90 was applied to the patient at the position of the tissue to be treated. The source may for example be an Iodine-125 implant. As a detector for the acquisition of the emitted radiation, respectively as an image providing element, in this embodiment a gamma camera 130 is used, which is connected via a cable 100 with a processing unit 110. The connection may in a further embodiment be wireless, for example an RF connection.

The camera 130 is freely movable in space and is equipped with a tracking marker 190. The position of the latter in space is detected by a tracking sensor 120, which is also connected to the processing unit 110. The tracking sensor 120 together with the processing unit 110 forms a tracking system 105. In embodiments, the tracking system may be an optical, electromagnetical, acoustical, mechanical, or RFID tracking system.

For the determination of the location of the target 90, the camera is moved freely over the body of the patient 50 and thereby receives the radiation emitted by the radiation source 90. At the same time, the tracking system 105 continuously detects the pose of the camera 130. Typically, also the orientation of the camera middle axis in space is recorded. By timely allocating the detected radiation values with the tracking data, a 3D image may be generated by applying image processing methods on the raw data (such as with freehand SPECT), and hence information may be derived about the geometrical location and the size of the radiation source 90.

The pose data of the gamma camera acquired by the tracking sensor 120 deliver, in conjunction with the detection data of the camera, a data basis for precisely determining the pose of the radiation source 90 with respect to the position of the tracking sensor 120. Thus, a position determination of the source 90 with respect to the tracking sensor 120 is possible. For determining if the absolute position of the radiation source 90, and the target tissue to be radiated, are at the set point position in the radiation path of the radiation therapy device 80, a geometrical relation of the tracking sensor 120 with the radiation therapy device 80 is required. This may for example be accounted for by the fact that the geometrical relation between the radiation therapy device 80, the radiation path of which and the position of the tracking sensor 120 are known and stored. By storing this information in a memory of the processing unit 110, for example during the first operation or calibration, the processing unit may on this basis align the pose of the radiation source 90 with the stored geometric data.

In embodiments, the tracking sensor 120 detects, aside from the camera pose, also further aspects of the radiation scenario, in particular the radiation therapy device 80 and the treatment couch 60, which are equipped with own tracking markers 192, 194 each. In this manner, the processing unit 110 may calculate a direct geometrical relation between the components detector (camera 130), maybe treatment couch 60 and radiation therapy device 80. The characteristic geometrical data of the radiation therapy device 80, in particular the beam path with respect to the outer dimensions of the device, may be stored in a memory of the processing unit 110. In this case, the processing units may directly calculate the correction value k by using the tracking data of all components in conjunction with the data acquired by the camera, which was processed into an image. With this correction value, the patient positioning device 70 may directly be controlled via an interface.

In embodiments, the calculated pose of the target tissue region may be transmitted via a (RF-) interface 73 to a control unit 112 (not displayed) of the radiation therapy device 80. The software for calculating the correction value k and for the respective control of the patient positioning device 70 is in this case provided in the control unit 112, differently to the previous examples. In embodiments the correction value k, as far as it can be displayed graphically, may be displayed on an optical display 180, if applicable as an overlay with a visual camera picture of the patient. In this manner, a control via an operator is possible. The repositioning of the patient may also be carried out by a manual action of the operator. Alternatively or additionally, an acoustical signal or the activation/deactivation of an interlock may be provided, for example when a movement of the patient occurs during radiation therapy. This may be carried out by the processing unit 110 or the radiation therapy device 80.

The correction value k serves as the basis for a correction the position of the patient 50, and thereby also of the radiation source 90 and the target tissue. In the most simple case, k is a two-dimensional vector, which indicates, into which direction and for which distance and direction the patient has to be moved in an x-y-plane (see FIG. 1) in order to position the target tissue in the beam path 82 of the radiation therapy device. In embodiments, k is a vector having three or more dimensions, wherein typically also a displacement component in a direction of the z coordinate (height position of the patient) is provided.

In embodiments, k may also be a matrix, for example a rigid 4×4 transformation matrix. It may for example also include a rotation, for example in the case that the position of the structure to be radiated in the body was altered, which could not be compensated by a mere translatory movement in x, y, z. In embodiments, k may also be a deformation field. This may for example be useful for correcting size differences of the target tissue (e.g. a tumor) in comparison to an image taken during a previous radiation fraction/session. For calculation of this deformation field, one can use methods for deformable registration such as non-parametrical registration methods, parametrical registration methods (e.g. with B-splines), curvature registration, demons registration, diffeomorphic demons registration, symmetric forces demons registration, level set motion registration, PDE deformable registration, etc. In general, in the embodiments described herein in it is assumed that the radiation therapy device, respectively the type of radiation therapy are of the type wherein the beam may be adjusted such that its cross section is basically identical to the cross-section of the target tissue to be radiated. At the same time, this means that there is a bijective pose of the target tissue or of the radiation source 90 in relation to the beam path 82, at which the optimum effect of the radiation treatment is achieved, because the beam cross section covers the whole tumor while saving adjacent healthy tissue by means of the collimation.

In embodiments it is also possible that the beam cross-section of the radiation therapy device 80 is smaller than the cross sectional area of the target tissue to be radiated. If the target is not only an implanted target, but is completely radioactive because of the injection of a radioactive tracer substance, in this case the image recognition system described above may be used to acquire the target tissue as a three dimensional spatial structure by the positioning system. In this scenario, the positioning system may be used to scan a cross-sectional area of the tumor/the target tissue with the beam of the radiation therapy device, such that successively the whole area of the tumor is radiated. For this purpose, a boundarization or segmentation of the tumor to be radiated against other, lightly radiating regions in the body may be carried out.

In other embodiments, the cross-section of the beam of the radiation therapy device 80 is bigger than a cross-section of the target tissue to be radiated. In this case, the shape of the beam cross-section may be derived from the shape of the target via a multi-leaf collimator.

The ability for operation in real time of the system according to embodiments may be used for continuously monitoring the pose of the target tissue during radiation therapy, so that continuously a correction value k may be calculated. If it oversteps a certain boundary condition, which generally means that the pose of the target tissue differs too much from a set point pose, which means that the target is no longer in the beam path 82, several measures may be provided. Possible is an optical display, for example on a display 180, or on a different operation monitor of the radiation therapy device 80, and an acoustical warning. If there is no reaction for a longer time, or if the overstepping is too significant, also the processing unit 110 or the processing units 112 of the radiation therapy device may trigger an automatical interruption of the radiation (e.g. by activating/deactivating an interlock). Alternatively, the processing unit 110, 112 may cause an automatical re-adjustment of the patient position via an order to to the patient positioning device 70 via interface 72.

In embodiments, a tracking element 196 is mounted to the patient, and the tracking system is configured to achieve tracking element coordinates, which provide a pose of the tracking element 196. In embodiments, a surface 86 of the treatment couch is provided with weight sensors for detecting a weight distribution of the person 50 to be imaged, when the person is lying on the surface. This may, amongst other factors, be used for additionally determining the movements of the patient. In embodiments the system further includes a surface determination system for localizing a body surface of the person to be imaged; preferably by using a tracked instrument for scanning the surface, wherein the instrument is a handheld gamma sonde, a hand gamma camera 130, a time of flight camera, a stereoscopic camera, a laser scanner, or an arbitrary combination of the former.

FIG. 2 shows a further embodiment. Differently to FIG. 1, there is no hand gamma camera 130 as an image providing detector, but to fixed detectors 150, 160 positioned left and right of the body of the patient (in the drawing in front of and behind the person, transparent for illustrational purposes). Via two radiation detectors 150, 160, also an image providing, space-resolved acquisition of the radiation field of the radiation source 90 in the patient 50 is achieved. The detectors are connected with the processing unit 110 (not shown). As the detectors are fixed, their spatial relation to the radiation therapy device 80 and its beam path are known, and are achievable by a one-time calibration. Hence, in this example no tracking system is required for setting the detected position of the radiation source into relation with the beam path 82 of of the radiation therapy device 80.

FIG. 3 shows a section of the detector arrangement 150, 160 of the system shown in FIG. 2. Those detectors may together form a PET detector or be two gamma cameras. Alternatively the detectors may each be the diffusion detector of a Compton camera, which can work together with an absorption detector (not shown).

FIG. 4 shows a detector arrangement of a system according to a further embodiment. Thereby, a locally fixed detector 160 aside to the patient is combined with a freely movable detector 130, which may be hand guided, but can also be controlled by a robot arm (not shown) via processing unit 110. Possible detector combinations are for example two fixed gamma cameras which are directed to a body part of interest, 1 PET detector, which includes two mounted PET plates, a fixed gamma camera and a hand guided tracked non-image providing detector (such as a gamma sonde), 2 freely tracked mini gamma cameras, etc.

It is obvious to the skilled person that a variety of combinations of different detector types may be applied in order to achieve a spatially resolved, image providing detection. For example, also two freely movable detectors of the type of the hand gamma camera 130 are possible.

FIG. 5 shows a schematic procedure of a method for determining the position of radioactively marked tissue of the patient during radiation therapy with a radiation therapy device according to embodiments. In a step 500 a radioactive radiation source is applied to the patient into a target tissue region to be radiated. In a step 510 the image providing detection of an emitted radiation distribution of the radiation source is carried out, wherein the patient is in a treatment position. In a step 530 follows the calculation of a pose of the target tissue region or the radiation source using the detected radiation. In a step 540 a correction value k is calculated as the difference between the calculated pose of the target tissue region and a setpoint value.

1. A system for the determination of a position of a target tissue of a patient during radiation therapy with a radiation therapy device, comprising:
at least one detector for the imaging of a radiation distribution of a radioactive source marking a target tissue region,
a processing unit, adapted for calculating a pose of the target tissue region from measurement data of the at least one detector, as well as for calculating a correction value as a difference between the calculated pose and comparative data,
an interface for transmitting data on the calculated pose of the target tissue region and the correction value from the processing unit to a radiation therapy device or to a patient positioning device.

2. The system according to claim 1, wherein the processing unit is adapted to calculate a the shape of the marked tissue from the measurement data.

3. The system according to claim 1, wherein the correction value is a vector with at least three dimensions, a matrix, and/or a deformation field.

4. The system according to claim 1, further comprising a tracking system for acquiring a pose of the at least one detector and/or the radiation therapy device, preferably an optical, electromagnetic, acoustic, mechanical or RFID tracking system.

5. The system according to claim 1, further comprising an output unit for displaying the correction value-graphically and/or acoustically, which is one of: a the vector with at least three dimensions, a matrix, and a deformation field.

6. The system according to claim 1, further comprising a patient positioning device for changing the patient's position of the patient according to the correction value.

7. The system according to claim 1, wherein the determination of the correction value is carried out in real time during a radiation therapy session, and wherein, while a boundary condition of the correction value is overstepped, at least one of the following is carried out:
signaling of the overstepping of the boundary condition to an operator,
automatically interrupting the radiation therapy.

8. The system according to claim 1, wherein the at least one detector is a PET detector, a SPECT detector, a freehand SPECT detector, a Compton camera, or a gamma camera.

9. The system according to claim 1, comprising at least two radiation detectors, having one of the following properties:
both are fixed,
one is movable, one is fixed,
both are movable.

10. The system according to claim 1, further comprising at least one of the following:
a tracking element to be mounted on the patient to be imaged, wherein the tracking system is configured to achieve tracking element coordinates, which provide a pose of the tracking element,
a surface with weight sensors for detecting a weight distribution of the patient to be imaged when lying on the surface,
a body surface determination system for localizing a body surface of the patient to be imaged, preferably via a guided instrument for scanning the surface, wherein the instrument is a handheld gamma camera, a time-of-flight-camera, a stereoscopic camera, a laser scanner, or any combination of the former.

11. A method for the determination of a position of a target tissue of a patient during radiation therapy with a radiation therapy device, comprising:
applying a radioactive radiation source into the target tissue to be therapeutically radiated for marking it,
imaging an emitted radiation distribution of the radiation source, with at least one detector, while the patient is in a treatment position,
calculation of a pose of the target tissue region using the detected radiation,
calculation of the correction value as a difference between the calculated pose of the target tissue region and a setpoint value of the pose.

12. A method according to claim 11, wherein 3D information about a shape and dimensions of the radioactively marked tissue is derived from the detected radiation measurements.

13. A method according to claim 12, wherein the 3D information is acquired by applying image processing methods on the detected radiation measurements.

14. The A method according to claim 11, further comprising at least one of the following:
communicating the calculated correction value to an operator of the radiation therapy device, preferably in acoustical and/or optical form,
activating an interlock for interrupting the radiation therapy.

15. The method according to claim 11, further comprising:
changing the relative position between the patient and the radiation therapy device in such a way that the target tissue region is located at a defined setpoint.

16. The method according to claim 11, wherein the application of the radioactive radiation source comprises at least one of the following:
implanting at least one target comprising a radioactive substance,
injecting a radioactive fluid into the patient's body.

17. The method according to claim 11, wherein the determination of the correction value is carried out in real time during radiation therapy treatment, and wherein, when a boundary condition for the correction value is overstepped, at least one of the following is carried out:
a. communicating the fact that a boundary condition of the correction value is overstepped to an operator operating the radiation therapy device,
b. automatically interrupting the radiation therapy treatment.

18. The method according to claim 11, wherein the correction value is one of: a vector with at least three dimensions, a matrix, and a deformation field.

19. The method according to claim 11, wherein the at least one detector is movable, and wherein its position is detected via one of: optical, electromagnetic, acoustic, mechanical, and RF tracking.
